(19) United States
(12) Patent Application Publication
(10) Pub. No.: US 2023/0381240 A1
(43) Pub. Date: Nov. 30, 2023

(54) COMPOSITION FOR BONE REGENERATION CONTAINING HYDROGEL AND STEM CELL SPHEROID, AND USE THEREOF

(71) Applicant: SELJIN CO.LTD., Seongnam-si (KR)

(72) Inventors: Jeong Gyun KIM, Cheongju-si (KR); Daye LEE, Seoul (KR); Wan Kyu KO, Suwon-si (KR); Seong Jun KIM, Goyang-si (KR); Gong Ho HAN, Seoul (KR); Su Bin CHO, Seoul (KR)

(73) Assignee: SELJIN CO.LTD., Seongnam-si (KR)

(21) Appl. No.: 17/816,998

(22) Filed: Aug. 2, 2022

(30) Foreign Application Priority Data
May 26, 2022 (KR) 10-2022-0064825

(51) Int. Cl. A61K 35/28; A61K 31/78; A61K 31/722; A61K 31/728; A61K 35/545; A61P 19/08; A61K 47/69; A61K 9/00

(52) U.S. Cl. CPC A61K 35/28; A61K 31/78; A61K 31/722; A61K 31/728; A61K 35/545; A61P 19/08; A61K 47/6903; A61K 9/0019

(57) ABSTRACT

The current disclosure relates to a composition for promoting bone regeneration, preventing or treating bone diseases using a composition containing a hydrogel and a stem cell spheroid and a use thereof, and a method for preparing the same, wherein the composition for bone regeneration has the effect of promoting bone regeneration of injured bone tissue and alleviating pain while promoting the differentiation of the injected mesenchymal stem cells into osteoblasts and osteocytes at the same time, and thus, can be used for the prevention or treatment of bone diseases.

### CROSS-REFERENCE TO RELATED APPLICATIONS

The present application claims the benefit of Korean Patent Application No. 10-2022-0064825, filed May 26, 2022, contents of which are incorporated herein by reference in its entirety.

### FIELD OF THE INVENTION

The disclosure is related to a composition for bone regeneration comprising a hydrogel and a stem cell spheroid, and method of for preventing or treating bone diseases.

### BACKGROUND OF THE INVENTION

Vertebral compression fractures in osteoporotic patients are generally treated with vertebroplasty, in which polymethyl methacrylate (PMMA) bone cement is injected into the spine to polymerize and harden to stabilize the fracture. As such, polymethyl methacrylate (PMMA) is a material widely used as bone cement and is mainly injected into the spine and hip joint areas as a filler for procedures on vertebral fractures with relatively few side effects. Filling the defect site with bone cement to minimize fractures is very effective in relieving severe pain. However, due to the inadequate strength of the injected bone cement, side effects, such as injury to the surrounding vertebrae, exfoliation of bone tissue in vivo, and blocking of resorption, are continuously emerging, hence the development of alternative materials is required.

In order to overcome such limitations, a technique for using a mixture of polymethyl methacrylate (PMMA) and an aqueous polymer gel has been suggested. A polymer gel, such as a hydrogel, acts as a pore-forming phase in which the gel phase dissolves or decomposes to form pores throughout the entire material. This is injected in vivo to maintain a hydrate environment to play the role of providing a scaffold in the form of a cushion and increase cell filtration.

The disclosure aims to suggest a more fundamental treatment for bone diseases by inducing bone tissue regeneration by mixing a hydrogel and a spheroid with the conventional polymethyl methacrylate (PMMA).

### SUMMARY OF THE INVENTION

One embodiment of the disclosure provides a composition and a method for bone regeneration, comprising polymethyl methacrylate (PMMA), a hydrogel, and a stem cell spheroid.

Another embodiment of the disclosure provides a pharmaceutical composition and a method for preventing or treating bone diseases, comprising polymethyl methacrylate (PMMA), a hydrogel, and a stem cell spheroid.

Yet another embodiment of the disclosure provides a method for preparing a composition for bone regeneration, wherein the method comprises a step for mixing glycol chitosan and oxidized hyaluronate in a weight ratio of 1 to 10:1 to obtain a hydrogel; and a step for mixing polymethyl methacrylate (PMMA) and a stem cell spheroid in the obtained hydrogel.

### BRIEF DESCRIPTION OF THE DRAWINGS

FIG. 1 is a diagram schematically showing the configuration of a composition for bone regeneration injected into a site where bone degeneration or bone disease has progressed.

FIG. 2A, FIG. 2B, FIG. 2C, and FIG. 2D are scanning electron microscope (SEM) photographs of PM gels in which a hydrogel and polymethyl methacrylate (PMMA) are mixed with different volume ratios according to an embodiment.

FIG. 3A is a schematic diagram and FIG. 3B is a photograph taken with an optical microscope showing that the seeded stem cells have been made in the form of spheroids according to an embodiment.

FIG. 4A and FIG. 4B are graphs showing the rheological characteristics of PM gel in which a hydrogel and polymethyl methacrylate (PMMA) are mixed with different volume ratios according to one embodiment.

FIG. 5 is a photograph showing the results of a test for cytotoxicity of PM gel in which a hydrogel and polymethyl methacrylate (PMMA) are mixed with different volume ratios according to one embodiment.

FIGS. 6A-6F are graphs showing the expression levels of osteoblasts and osteocytes for checking the osteogenic differentiation of stem cell spheroids according to an embodiment.

FIG. 7 is a diagram showing a schematic diagram of an animal experiment for analyzing the in vivo activity of a composition for bone regeneration (PMMS) according to an embodiment.

FIG. 8A is a micro-CT photograph of the femur of a normal rat without removing the ovary and without injury, FIG. 8B is a micro-CT photograph of the femur of a rat with the ovary removed but without injury.

FIGS. 9A-9D are micro-CT photographs of a composition for bone regeneration injected into the femur according to an embodiment; Injury: OVX+injury after four weeks (FIG. 9A), PMMA: OVX+injury after four weeks+PMMA (FIG. 9B), PM gel: OVX+injury after four weeks+PM gel (hydrogel:PMMA=8:2) (FIG. 9C), PMMS: OVX+injury after four weeks+PM gel (hydrogel:PMMA=8:2)+spheroids (FIG. 9D).

FIG. 10A is a qualitative analysis image and FIG. 10B is a quantitative analysis graph of the degree of recovery through the pain marker TRPV1 of the composition for bone regeneration according to an embodiment; Injury: OVX+injury after four weeks, PMMA: OVX+injury after four weeks+PMMA, PM gel: OVX+injury after four weeks+PM gel (hydrogel:PMMA=8:2), PMMS: OVX+injury after four weeks+PM gel (hydrogel:PMMA=8:2)+spheroids

### DETAILED DESCRIPTION OF THE INVENTION

One aspect of the disclosure provides a composition for bone regeneration comprising polymethyl methacrylate (PMMA), a hydrogel, and a stem cell spheroid:
wherein,
the hydrogel comprises glycol chitosan and oxidized hyaluronate, and the glycol chitosan and oxidized hyaluronate is mixed in a weight ratio of 1 to 10:1;
the hydrogel solution and polymethyl methacrylate (PMMA) is mixed in a volume ratio of 1 to 5:1; and
the stem cells is selected from among human-derived pluripotent stem cells (PSC), embryonic stem cells (ESC), mesenchymal stem cells (MSC), adult stem cells (ASC), induced pluripotent stem cells (iPSC), or a combination thereof;
wherein, the stem cells may be mesenchymal stem cells (MSC).

In one embodiment, the composition may further comprise an osteoinductive material, an osteoconductive material, an osteogenic material, an osteopromotive material, an anti-osteoporotic material, or an osteophilic material.

In one embodiment, the composition may promote the growth of cortical bone.

Another aspect of the disclosure provides a pharmaceutical composition for preventing or treating bone diseases comprising polymethyl methacrylate (PMMA), a hydrogel, and a stem cell spheroid.

In one embodiment, the bone disease may be selected from the group consisting of osteoporosis, compression fracture, lumbar herniated intervertebral disc, degenerative arthritis, rheumatoid arthritis, Paget's disease, osteomalacia, osteosclerosis, and bone tumor.

Another aspect of the disclosure provides a method for preparing a composition for bone regeneration, wherein the method comprises a step for mixing glycol chitosan and oxidized hyaluronate in a weight ratio of 1 to 10:1 to obtain a hydrogel; and a step for mixing polymethyl methacrylate (PMMA) and a stem cell spheroid in the obtained hydrogel.

In one embodiment, the hydrogel solution and polymethyl methacrylate (PMMA) may be mixed in a volume ratio of 1 to 5:1.

In one embodiment, the stem cells may be composed of human-derived pluripotent stem cells (PSC), embryonic stem cells (ESC), mesenchymal stem cells (MSC), adult stem cells (ASC), induced pluripotent stem cells (iPSC), or a combination thereof.

In one embodiment, the stem cells may be mesenchymal stem cells (MSC).

In one embodiment, may further comprise a step for adding an osteoinductive material, an osteoconductive material, an osteogenic material, an osteopromotive material, an anti-osteoporotic material, or an osteophilic material.

An embodiment of the disclosure provides a method for promoting bone regeneration, preventing or treating bone disease comprising:
administering an effective amount of a pharmaceutical composition comprising:
polymethyl methacrylate (PMMA), a hydrogel, and a stem cell spheroid:
wherein,
the hydrogel comprises glycol chitosan and oxidized hyaluronate, and the glycol chitosan and oxidized hyaluronate is mixed in a weight ratio of 1 to 10:1;
the hydrogel solution and polymethyl methacrylate (PMMA) is mixed in a volume ratio of 1 to 5:1; and
the stem cells is selected from among human-derived pluripotent stem cells (PSC), embryonic stem cells (ESC), mesenchymal stem cells (MSC), adult stem cells (ASC), induced pluripotent stem cells (iPSC), or a combination thereof;
wherein, the stem cells may be mesenchymal stem cells (MSC).

In one embodiment, the composition is injected into a site where bone regeneration needs to be promoted or bone disease has progressed.

According to a composition for bone regeneration and a method for preparing the same, the composition for bone regeneration has the effect of promoting bone regeneration of injured bone tissue and alleviating pain while promoting the differentiation of the injected mesenchymal stem cells into osteoblasts and osteocytes at the same time, and thus, it can be used for the prevention or treatment of bone diseases.

One aspect provides a composition for bone regeneration prepared by mixing polymethyl methacrylate (PMMA), a hydrogel, and a stem cell spheroid and a method for preparing the composition.

In one embodiment, the hydrogel comprises glycol chitosan and oxidized hyaluronate, and glycol chitosan and oxidized hyaluronate may be mixed in a weight ratio of 1 to 10:1. For example, the glycol chitosan and oxidized hyaluronate may be mixed in a weight ratio of 1 to 10:1, 1 to 9:1, 1 to 8:1, 1 to 7:1, 2 to 10:1, 2 to 8:1, 3 to 10:1, 3 to 8:1, 3 to 8:1, 4 to 10:1, 4 to 8:1, 5 to 10:1, 5 to 9:1, 6 to 10:1, or 6 to 8:1. In this case, if the mixing ratio of glycol chitosan and oxidized hyaluronate is less than said range or exceeds said range, glycol chitosan and oxidized hyaluronate do not get sufficiently crosslinked, forming fluid in a state of liquid with low viscosity, which presents a problem in that a gel in a solid or semi-solid state is not formed. Therefore, when the hydrogel is injected into the injured site, the rate of absorption into the body is very fast or it is easily dissolved in the body, hence its efficacy as a therapeutic agent may not be exhibited.

According to one embodiment, the hydrogel may provide an empty space such that the cells can grow at the location where polymethyl methacrylate (PMMA) and stem cell spheroids are injected and the site adjacent thereto. Specifically, the cells may comprise stem cell spheroids, progenitor-cells differentiated from stem cell spheroids, such as mesenchymal stromal cells, somatic cells fully differentiated from stem cell spheroids, for example, chondrocytes, osteocytes, and adipocytes.

Specifically, the density of the polymethyl methacrylate (PMMA) may be 1.15 to 1.19 g/cm3.

In one embodiment, the hydrogel and polymethyl methacrylate (PMMA) may be mixed in a weight ratio of 1 to 10:1. For example, PM gel may be formed by mixing the hydrogel and polymethyl methacrylate (PMMA) at a weight ratio of 1 to 10:1, 1 to 9:1, 1 to 8:1, 1 to 7:1, 2 to 10:1, 2 to 8:1, 3 to 10:1, 3 to 8:1, 4 to 10:1, 4 to 8:1, 5 to 10:1, 5 to 8:1, 6 to 10:1, or 6 to 8:1.

In one embodiment, the hydrogel solution and polymethyl methacrylate (PMMA) may be mixed in a volume ratio of 1 to 10:1. For example, PM gel may be formed by mixing the hydrogel solution and polymethyl methacrylate (PMMA) at a volume ratio of 1 to 10:1, 1 to 9:1, 1 to 8:1, 1 to 7:1, 2 to 10:1, 2 to 8:1, 3 to 10:1, 3 to 8:1, 4 to 10:1, 4 to 8:1, 5 to 10:1, 5 to 8:1, 6 to 10:1, or 6 to 8:1.

In an embodiment, the PM gel may have a storage modulus (G′) of 4000 to 170000 Pa, 4000 to 162000 Pa, 4000 to 71000 Pa, 4000 to 20000 Pa, 29000 to 170000 Pa or 29000 to 162000 Pa, 29000 to 71000 Pa, 55500 to 170000 Pa, 55500 to 162000 Pa or 55500 to 71000 Pa.

In one embodiment, the measured storage modulus (G′) of the PM gel may have a higher value the greater the volume ratio of polymethyl methacrylate (PMMA) to hydrogel.

In one embodiment, the PM gel may have a loss modulus (G″) of 1250 to 37200 Pa, 1250 to 12800 Pa, 1250 to 4300 Pa, 5600 to 37200 Pa, 5600 to 12800 Pa or 9300 to 37200 Pa.

The elastic modulus may be measured with a viscometer (e.g., rotating rheometer) at a compression rate of 2 mm/min when vibration is applied from 0.1 Hz to 10 Hz.

According to one embodiment, the hydrogel or PM gel may further comprise a physiologically active substance in one embodiment. Examples of the physiologically active substances may include anti-inflammatory drugs, anti-cancer drugs, contrast agents, hormone drugs, anti-hormone drugs, vitamin supplements, calcium agents, mineral preparations, saccharides, organic acid preparations, protein amino acid preparations, antidotes, enzyme preparations, metabolic agents, diabetes combination agents, tissue regeneration agents, chlorophyll agents, color formulations, tumor drugs, oncology drugs, radiopharmaceuticals, tissue cell diagnostic agents, tissue cell therapeutic agents, antibiotic agents, antiviral agents, complex antibiotic agents, chemotherapeutic agents, vaccines, toxins, toxoids, anti-toxins, leptospirin serums, blood products, biologic agents, analgesics, immunogenic molecules, antihistamines, allergy drugs, non-specific immunogenic agents, anesthetics, stimulants, psychoneurotic agents, etc. In a specific embodiment, the physiologically active substances may be a therapeutic agent for spinal cord injury, for example, an anti-inflammatory agent, more specifically, ursodeoxycholic acid.

In the present specification, the term “stem cells” refers to cells having the ability for differentiating into various types of body tissues. In addition, it refers to cells capable of differentiating into various tissue cells when conditions are set in an undifferentiated state.

According to one embodiment, the stem cells are not limited to human-derived pluripotent stem cells (PSC), embryonic stem cells (ESC), mesenchymal stem cells (MSC), adult stem cells (ASC), induced pluripotent stem cells (iPSC), or a combination thereof. More specifically, the stem cells may be mesenchymal stem cells.

The “mesenchymal sterm [sic: stem] cell (MSC)” is a stem cell having multipotency and self-renewal ability and refers to a stem cell capable of differentiating into various cells, for example, adipocytes, chondrocytes, and osteocytes.

In the present specification, the term “differentiation” refers to a phenomenon in which the structure or function becomes specialized while cells divide, proliferate, and grow, that is, the form or function of cells, tissues, etc. of organisms change in order to perform tasks given to each of them.

In the present specification, the term “spheroid” refers to a three-dimensionally modeled cellular structure.

The stem cell spheroid may have the effect of inducing differentiation from stem cells into osteocytes. Furthermore, it may have the effect of inhibiting the hypertrophy and dedifferentiation of osteocytes. When the composition for bone regeneration including the spheroid is injected into bone tissue, it may have the effect that osteocytes are differentiated, and bone cells are regenerated.

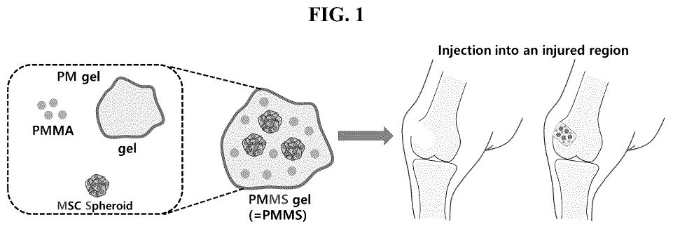

The composition for bone regeneration may be injectable into a site where bone disease has progressed. Specifically, it may serve to fill a region with bone defects by being injected through a surgical method into a patient with decreased bone density or advanced bone degeneration. In one embodiment, as shown in FIG. 1, the composition for bone regeneration may be injected into a bone region.

The composition for bone regeneration may be useful for repairing orthopedic symptoms. As a non-limiting example, it may be injected into the vertebral body for the treatment of vertebral fractures, injected into long or flat bone fractures to enhance fracture repair or to stabilize fracture fragments, or injected into intact osteoporotic bone to improve strength. The composition is capable of providing an elastic modulus closer to bone modulus compared to conventional bone cement, and at the same time, inducing bone regeneration to mimic the properties of normal bone and support weight load. Due to such enhanced weight load capability, the composition can provide scaffold support in connection with various types of spine fractures, can be used to strengthen and prevent tibial plateau reconstruction, wrist fracture reconstruction, heel bone reconstruction, and can be used for traumatic fractures such as osteoporotic vertebral compression fractures and tibial plateau fractures.

The composition for bone regeneration may further comprise an osteoinductive material, an osteoconductive material, an osteogenic material, an osteopromotive material, an anti-osteoporotic, or an osteophilic material.

Specifically, the “osteoinductive material” refers to a material that induces the formation of interosseous cells (that is, cells capable of forming new bone or bone material) by inducing mitogenesis of undifferentiated perivascular mesenchymal cells. The “osteoconductive material” refers to a material that facilitates the formation or new bone or bone material into vascular penetration and certain passive trellis structures. This may include those exhibiting osteoinductive, osteoconductive, osteogenic, osteopromotive, or osteophilic activity among various known compounds, minerals, proteins, etc.

Specifically, osteoinductive and osteoconductive materials may include demineralized bone matrix (DBM), bone morphogenic protein (BMP), transforming growth factor (TGF), fibroblast growth factor (FGF), insulin-like growth factor (IGF), platelet-derived growth factor (PDGF), epidermal growth factor (EGF), vascular endothelial growth factor (VEGF), peptides, inorganic bone mineral (ABM), vascular permeability factor (VPF), cell adhesion molecule (CAM), calcium aluminate, hydroxyapatite, coralline hydroxyapatite, alumina, zirconia, aluminum silicate, calcium phosphate, tricalcium phosphate, brushite (dicalcium phosphate dihydrate), tetra-calcium phosphate, octa-calcium phosphate, calcium sulfate, polypropylene fumarate, pyrolytic carbon, bioactive glass, porous titanium, porous nickel-titanium alloy, porous tantalum, sintered cobalt-chromium beads, ceramics, collagen, autologous bone, allogenic bone, xenogenic bone, coralline, and derivatives or combinations thereof, or other complex materials containing calcium or hydroxyapatite structural elements and biologically produced, but are not limited thereto.

Specifically, an osteogenic material may comprise osteogenic proteins (e.g., OP-1, OP-2, or OP-3), transforming growth factor-α, transforming growth factor-β (e.g., (31, (32, or (33), LIM mineralization protein (LMP), ovulation-inducing factor (OIF), angiogenin, endothelin, growth differentiation factor (GDF), ADMP-1, endothelin, hepatocyte growth factor and keratinocyte growth factor, osteogenin (bone morphogenetic protein-3), heparin binding growth factor (HBFG) (e.g., HBGF-1 and HBGF-2), interleukins (IL) including IL-1 to -6, colony stimulating factors (CSF) including CSF-1, G-CSF, and GM-CSF, epidermal growth factor (EGF), insulin-like growth factor (e.g., IGF-I and -II), demineralized bone matrix (DBM), cytokines, osteopontin, and osteonectin, but are not limited thereto.

Additionally, additives may be added to adjust the properties of the prepared composition. Specifically, it may include proteins, radiopaque agents, for example, strontium phosphate or strontium oxide, drugs, supportive or reinforcing filler materials, crystal growth controlling agents, viscosity controlling agents, pore-forming agents, antibiotics, antiseptics, growth factors, chemotherapeutic agents, bone resorption inhibitors, color changing agents, immersion liquid, carboxylate, carboxylic acid, α-hydroxy acids, metal ions, or mixtures thereof. Other examples may include materials that modulate coagulation time (e.g., pyrophosphate or sulfate), or increase injectability or cohesion (e.g., hydrophobic polymers such as collagen).

The composition for bone regeneration may induce new bon [sic: bone] and bone ingrowth and induce cortical bone thickness to be within a normal range.

Another aspect provides a pharmaceutical composition for preventing or treating bone disease comprising polymethyl methacrylate (PMMA), a hydrogel, and a stem cell spheroid.

The hydrogel, stem cell, and spheroid are as described above.

In the present specification, the term “bone disease” refers to diseases related to the bone that appear when bone density is lowered, or bone generation progresses. Examples of bone disease include osteoporosis, compression fracture, lumbar herniated intervertebral disc, degenerative arthritis, rheumatoid arthritis, Paget's disease, osteomalacia, osteosclerosis, and bone tumor, but are not limited thereto.

In the present specification, the term “pharmaceutical composition” may refer to a molecule or compound that provides some advantageous effects when administered to a subject. Advantageous effects may include enabling diagnostic decisions; improving a disease, symptom, disorder, or condition; reducing or preventing the onset of a disease, symptom, disorder, or condition; and generally responding to a disease, symptom, disorder, or condition.

The pharmaceutical composition may be administered orally or parenterally for clinical administration and may be used in the form of a general pharmaceutical formulation. Parenteral administration may refer to administration via routes other than oral administration such as rectal, intravenous, peritoneal, muscle, arterial, transdermal, nasal, inhalation, ocular, and subcutaneous administration. When the pharmaceutical composition of the disclosure is used as a drug, it may further contain one or more active ingredients exhibiting the same or similar functions.

The types of pharmaceutically active ingredients capable of delivering the active ingredient into a subject may comprise anticancer agents, contrast medium (dye), hormone agents, anti-hormonal agents, vitamin supplements, calcium agents, mineral preparations, saccharides, organic acid preparations, protein amino acid preparations, antidotes, enzyme preparations, metabolic agents, diabetes combination agents, tissue regeneration agents, chlorophyll agents, color formulations, tumor drugs, oncology drugs, radiopharmaceuticals, tissue cell diagnostic agents, tissue cell therapeutic agents, antibiotic agents, antiviral agents, complex antibiotic agents, chemotherapeutic agents, vaccines, toxins, toxoids, anti-toxins, leptospirin serums, blood products, biologic agents, analgesics, immunogenic molecules, antihistamines, allergy drugs, non-specific immunogenic agents, anesthetics, stimulants, psychoneurotic agents, low molecule weight compounds, nucleic acids, aptamers, antisense nucleic acids, oligonucleotides, peptides, siRNAs, micro RNAs, etc.

When formulating the pharmaceutical composition, it is prepared using a diluent or excipient, such as commonly used fillers, extenders, binders, wetting agents, disintegrating agents, and surfactants. Formulations for parenteral administration include sterile aqueous solutions, nonaqueous solutions, suspensions, emulsions, freeze-dried preparations, and suppositories. For nonaqueous solutions and suspensions, propylene glycol, polyethylene glycol, vegetable oils such as olive oil, and injectable esters such as ethyl oleate may be used. For the base of the suppository, Witepsol, macrogol, Tween 61, cacao fat, liurin fat, and glycerogelatin may be used.

In addition, the pharmaceutical composition may be used by mixing with various allowed carriers such as physiological saline and organic solvents, and in order to increase stability or absorbency, carbohydrates such as glucose, sucrose, or dextran, antioxidants such as ascorbic acid or glutathione, chelating agents, low molecular weight proteins, or other stabilizers may be used as medicaments.

In addition, the pharmaceutically effective amount and effective dosage of the pharmaceutical composition may vary depending on the formulation method, administration method, administration time and/or administration route of the pharmaceutical composition. In addition, it may vary depending on various factors, such as the type and degree of response to be achieved by administration of the pharmaceutical composition, the type of subject to be administered, age, weight, general health condition, symptoms or degree of disease, gender, diet, excretion, components of drugs and other compositions used simultaneously or separately on the relevant subject, and similar factors widely known in the medical field. A person skilled in the art can easily determine and prescribe an effective dosage for the desired treatment. As for the administration of the pharmaceutical composition according to the disclosure, it may be administered once a day, or it may be divided and administered several times. Therefore, said dosage does not limit the scope of the disclosure in any way. The dosage of the pharmaceutical composition may be 1 ug/kg/day to 1,000 mg/kg/day per day.

The subject may be a mammal, for example, a human, cow, horse, pig, dog, sheep, goat, or cat. The subject may be a subject in need of treatment of bone disease.

The pharmaceutical composition of the disclosure can promote the regeneration of bone tissue by inducing specific differentiation of stem cell spheroids into osteocytes. Therefore, as with the conventional injection of polymethyl methacrylate (PMMA) bone cement, it can reduce side effects such as injury to the surrounding vertebrae and exfoliation of bone tissue in vivo, and at the same time, induce bone regeneration around the injected site to enable fundamental treatment of diseases related to decreased bone density or bone degeneration.

Hereinafter, preferred embodiments will be presented to aid in understanding of the disclosure. However, the following embodiments are only provided so that the disclosure can be understood more easily, and the contents of the disclosure are not limited by the following embodiments. Since the embodiments may apply various modifications, the embodiments are not limited to the embodiment disclosed below and may be implemented in various forms.

### Example 1. Preparation of PM Gel

As for polymethyl methacrylate (PMMA), Teknimed SPINE FIX® product from Teknimed was purchased and used. The PMMA was mixed with hydrogel to prepare PM gel. A hydrogel is a gel made by crosslinking glycol chitosan and oxidized hyaluronate. For the production of oxidized hyaluronate, hyaluronate (HA) (molecular weight (MW)=1,000 kDa) was purchased from Hymedix (Korea). Then, oxidized hyaluronate (oHA) was made by oxidizing using a method of stirring with sodium periodate (Sigma-Aldrich, USA) for 24 hours. Then, 1 ml of ethylene glycol (Sigma-Aldrich, USA) was added to neutralize sodium periodate that did not react with HA. Then, the solution was dialyzed for seven days using a dialysis membrane (Spectrum Spectra, molecular weight cut off (MWCO): 12-14K) and then freeze-dried (see Korean Published Patent 10-2021-0153788). Glycol chitosan was purchased from Sigma-Aldrich (USA), and 200 mg of the purchased glycol chitosan was dissolved in 10 ml of DPBS solution, 2% glycol chitosan (gC) and 3% oxidized hyaluronate (oHA) were mixed in a volume ratio of 9:1, and then, it was purified in the same manner as described above to prepare a glycol chitosan solution. A gel was produced by crosslinking the prepared glycol chitosan and oxidized hyaluronate by the method described in said patent.

PM gel was prepared by mixing the hydrogel prepared as described above and polymethyl methacrylate (PMMA) in volume ratios of 8:2, 7:3, and 6:4, respectively.

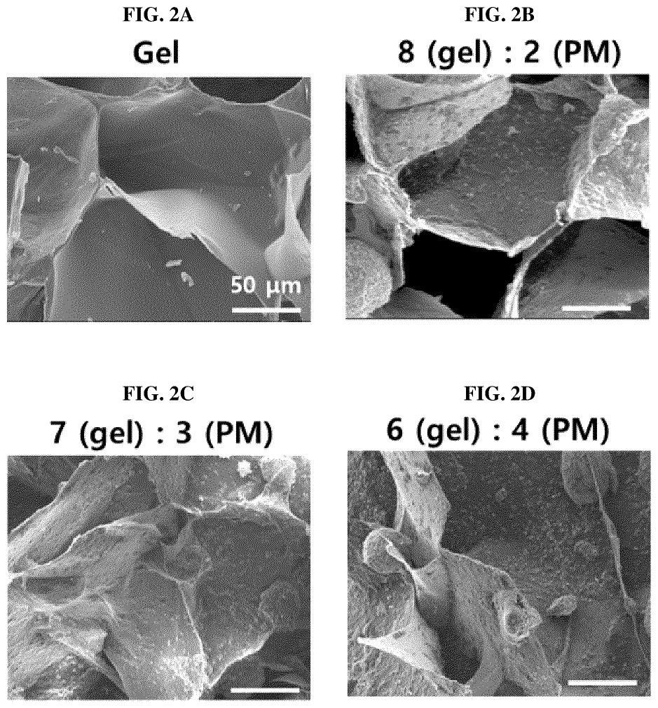

As shown in FIGS. 2A-2D, the shape of the surface of the PM gel prepared by mixing hydrogel and polymethyl methacrylate (PMMA) at a volume ratio of 8:2, 7:3, and 6:4, respectively, was confirmed through a screening electron microscope (SEM) (scale bar: 50 μm).

FIGS. 2A-2D is a scanning electron microscope (SEM) photograph of PM gel in which a hydrogel and polymethyl methacrylate (PMMA) are mixed with different volume ratios according to an embodiment.

### Example 2. Preparation of PMMS

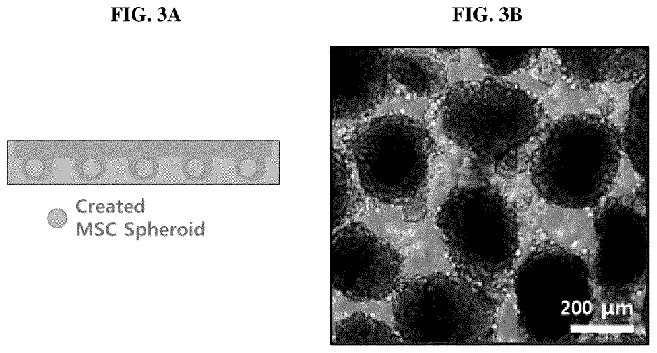

Bone marrow-derived MCSs were used and StemPro™ BM Mesenchymal Stem Cells were purchased from Thermo Fisher Scientific, USA. The purchased mesenchymal stem cells (MSC) were seeded on a dedicated plate (StemFIT 3D®, Korea) to be made in the form of mesenchymal stem cell spheroids (MSC spheroids). Specifically, 1 ml of mesenchymal stem cells (MSCs) per well was seeded at a concentration of 1×10⁶ cells/mL on a dedicated plate, the spheroids formed after 24 hours were used for the experiment, and the shape of its surface was confirmed using an optical microscope (Nicon, TS2, Japan), which was illustrated in FIG. 3 (scale bar: 200 μm).

FIG. 3A is a schematic diagram and FIG. 3B is a photograph taken with an optical microscope showing that the seeded stem cells have been made in the form of spheroids according to an embodiment of the disclosure.

### Experimental Example 1. Measurement of Rheological Characteristics of PM Gel

The rheological characteristics of PM gel prepared by mixing the hydrogel prepared in embodiment 1 and polymethyl methacrylate (PMMA) in volume ratios of 8:2, 7:3, and 6:4, respectively, were confirmed. After preparing 200 μL of gel and PM gel, strain-dependent storage modulus (G′) and loss modulus (G″) were measured using a rotating rheometer (AR-G2, TA Instruments, USA) at a compression rate of 2 mm/min from 0.1 until reaching 10 Hz.

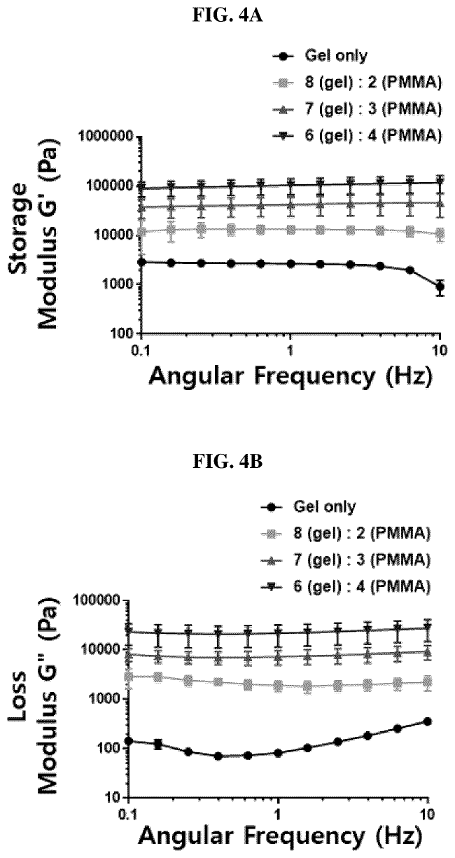

FIGS. 4A and 4B are graphs showing the rheological characteristics of PM gel in which a hydrogel and polymethyl methacrylate (PMMA) are mixed with different volume ratios according to one embodiment.

As shown in FIGS. 4A and 4B, in the case of PM gel in which the hydrogel prepared by the above method and polymethyl methacrylate (PMMA) are mixed in volume ratios of 8:2, 7:3, and 6:4, when the angular frequency was 1 (=when the X-axis value was 1), G′ increased in the order of 13386±2511 Pa, 42301±17874 Pa, and 103266±36884 Pa. When the X-axis value was 1, the G′ value of the gel was 2675±274 Pa. In addition, the G″ of PM gel mixed in the same volume ratio increased in the order of 1916±465 Pa, 7311±2332 Pa, and 21745±10191 Pa. When the X-axis value was 1, the G″ value of the gel was 83±6 Pa.

### Experimental Example 2. Analysis of Cell Viability of PM Gel

In order to confirm the cytotoxicity of PM gel, as a qualitative analysis of cell viability, the cells were separated into live cells (stained with green) and dead cells (stained with red) using a live and dead kit (Invitrogen, #L3224) and observed with a fluorescence microscope (×20, Olympus BX53F) to perform the analysis.

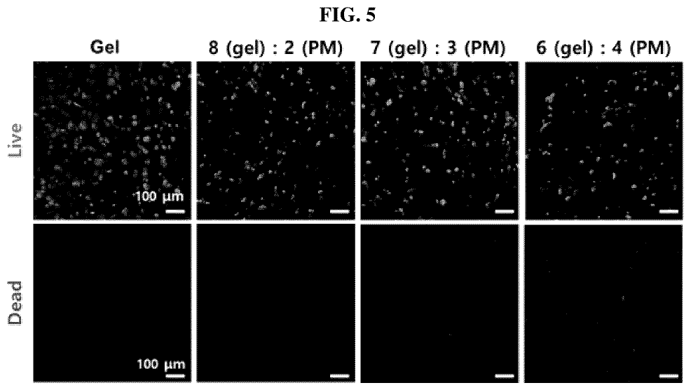

First, the purchased bone marrow-derived MSCs (Thermo Fisher, USA, #A15652) were cultured in a growth medium (GM) composed of MesenPRO RS™ medium (Thermo Fisher, USA, #12746012) supplemented with 2% fetal bovine serum (FBS, GIBCO) and 1% penicillin-streptomycin. Then, MSCs (2×10⁵) were mixed with PM gel in which a hydrogel and polymethyl methacrylate (PMMA) are mixed in volume ratios of 8:2, 7:3, and 6:4. Thereafter, the hydrogel containing the cells was seeded in a 48-well culture plate, incubated together with a growth medium for 48 hours to verify cytotoxicity, and observed under a fluorescence microscope, and this is illustrated in FIG. 5 (scale bar: 100 μm).

FIG. 5 is a photograph showing the results of a cytotoxicity test of PM gel in which a hydrogel and polymethyl methacrylate (PMMA) are mixed with different volume ratios according to one embodiment.

As shown in FIG. 5, no dead cells were observed in two groups including Gel and 8:2 PM gel, and it was seen that the dead cells appear gradually as it becomes 7:3 and 6:4.

### Experimental Example 3. Osteo-Differentiation Potency Analysis

In order to check the osteo-differentiation potency of stem cell spheroids, the expression levels of osteo-differentiation markers were measured using real time RT-PCR.

The osteo-differentiation experiment was conducted on a total of two groups including a group grown in a 2D form on a 35-pie plate (SPL, Korea) (n=3) and a group grown in a 3D spheroid form on a 35-pie plate (SPL, Korea) (n=3), and 1×10⁶ per plate was seeded, and the expression level of each osteo-differentiation marker was analyzed for each group after seven days, and for the osteo-differentiation induction medium, fetal bovine serum (FBS, GIBCO), penicillin (GIBCO), streptomycin (GIBCO), β-glycerol phosphate disodium salt hydrate (Sigma-Aldrich), ascorbic acid (Sigma-Aldrich), and dexamethasone (Sigma-Aldrich) added to Dulbecco's Modified eagle Medium (DMEM, GIBCO) were used.

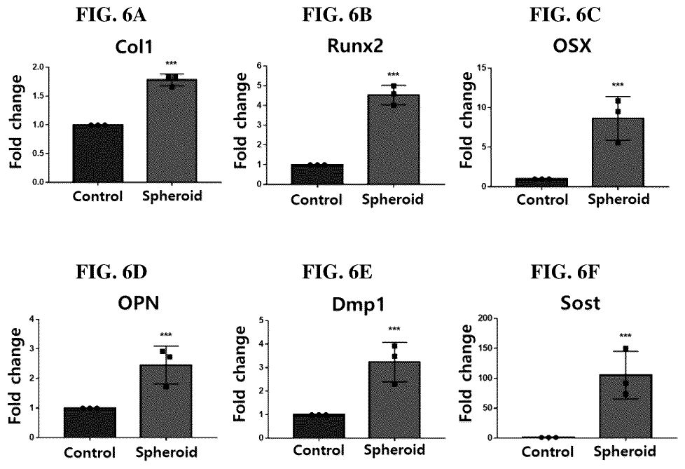

It was performed on Coll (Collagen Type 1), Run×2 (Runt-related Transcription Factor2), OSX (Osterix), OPN (Osteopontin), Dmp1 (Dentin matrix acidic phosphoproteinl), and Sost (Sclerostin), which are representative osteo-differentiation markers. Run×2 and OSX are representative key transcription factors for the differentiation of osteo-differentiation cells, and COL1 is an early osteo-differentiation marker. OPN is a late marker of osteoblast differentiation and an early marker of osteocytes, and Dmp1 and Sost are osteocyte markers. The expression level of the control group (=2D plate) was determined to be the reference, and the differentiation increase rate of the stem cell spheroid group was calculated by setting the reference value to 1, and the results have been illustrated in FIGS. 6A-6F.

FIGS. 6A-6F are graphs showing the expression levels of osteoblasts and osteocytes for confirming the osteo-differentiation of stem cell spheroids according to one embodiment.

As shown in FIGS. 6A-6F, it was confirmed that the expression level of osteo-differentiation markers in the stem cell spheroid group increased by at least 2 times and up to 100 times or more compared to the control group.

### Experimental Example 4. Activity Analysis of the Composition for Bone Regeneration (In Vivo)

The in vivo activity of the composition for bone regeneration (PMMS) has been analyzed as follows. The experiment was performed using 8-week-old female Sprague [sic:] Sprague-Dawley (SD) rats (200 to 230 g).

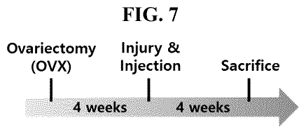

FIG. 7 is a drawing showing a schematic diagram of an animal experiment for analyzing the in vivo activity of a composition for bone regeneration (PMMS) according to one embodiment.

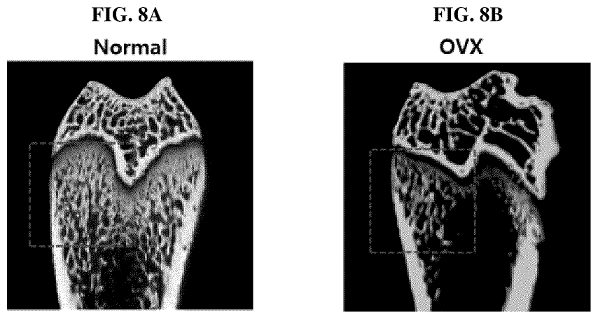

FIG. 8A is a micro-CT photograph of the femur of a normal rat without removing the ovary and without injury, and FIG. 8B is a micro-CT photograph of the femur of a rat with the ovary removed but without injury.

In the in vivo experiment, a total of four groups were used with three animals in each group, and they were classified as follows. All four groups were modeled for osteoporosis by removing the ovaries, and four weeks after removing the ovaries, a circular trephine (FTS product, #18004-27, tip diameter: 2.7 mm) was dug into the femur to a depth of 2 mm to cause an injury by the method for creating an empty space as large as the corresponding space.

Group 1: After performing ovariectomy (OVX), the femur was injured but nothing was injected into the injured site (labeled ‘Injury’).

Group 2: After performing ovariectomy (OVX), the femur was injured and polymethyl methacrylate (PMMA) was injected into the injured site (labeled ‘PMMA’).

Group 3: After performing ovariectomy (OVX), the femur was injured and a hydrogel and polymethyl methacrylate (PMMA) were mixed in a volume ratio of 8:2 and injected into the injured site (labeled ‘PM gel’).

Group 4: After performing ovariectomy (OVX), the femur was injured, a hydrogel and polymethyl methacrylate (PMMA) were mixed in a volume ratio of 8:2, and stem cell spheroids were injected into the injured site (labeled ‘PMMS’).

### Experimental Example 4.1 Three-Dimensional Analysis (Micro CT)

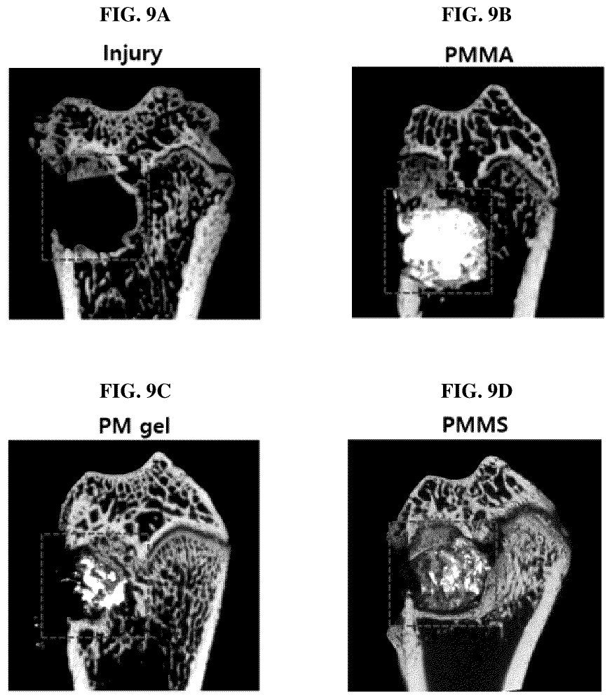

For three-dimensional analysis using micro-CT (μCT) equipment, immediately after sacrificing the animal, the image of the bone of the specimen for the femur of the specimen was photographed 512 times per specimen in DICOM (digital imaging and communication in medicine) files using μCT equipment called Quantum FX (Perkin Elmer, Waltham, MA, USA), and the 256th photo, which is the center part, was selected as a representative image and shown in FIGS. 9A-9D.

FIGS. 9A-9D are micro-CT photographs of a composition for bone regeneration injected into the femur according to an embodiment; Injury: OVX+injury after four weeks, PMMA: OVX+injury after four weeks+PMMA, PM gel: OVX+injury after four weeks+PM gel (hydrogel:PMMA=8:2), PMMS: OVX+injury after four weeks+PM gel (hydrogel:PMMA=8:2)+spheroids

As shown in FIGS. 9A-9D, the number of portions appearing gray (bone) was significantly increased in the PMMS group compared to the PMMA group that only appears white, which confirmed that the portions appearing gray (bone) increases even when compared to the PM gel group.

Therefore, it can be understood that the PMMS composition for bone regeneration according to an aspect promotes differentiation into osteoblasts and osteocytes, and at the same time, promotes bone regeneration, showing that it is useful for the recovery of injured bone tissue.

Experimental example 4.2 Analysis of the degree of pain recovery Analysis of the degree of cellular pain recovery was performed by using fluorescent staining to distinguish neuronal cells NeuN (stained with green) and normalized marker TRPV1 of nociceptor (stained with red) and observing them with a confocal microscope (LSM 880, Germany, ×20).

It was checked whether the expression of the pain marker TRPV 1 (transient receptor potential vanilloid 1) was reduced. Since the corresponding pain marker is expressed in nerve cells, it was checked by staining together with NeuN (Neuronal neuclei), which is a marker dedicated to nerve cells.

Nerve pain occurs in the signal transduction of injured nerve cells or sensory neurons. The corresponding nerve pain signal is transmitted to sensory neurons in the dorsal root ganglia (DRG).

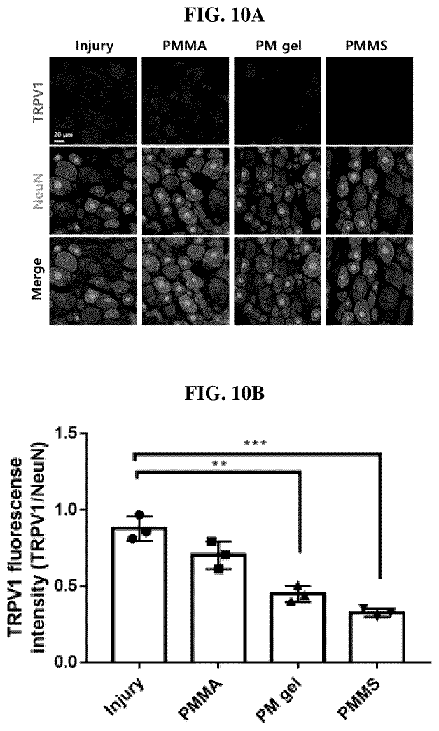

Specifically, the ovary was removed, the femur was injured four weeks later, and immediately after, PMMA or a mixture suitable for each group (n=3) was administered, and then, the rats were perfused again after four weeks to extract DRG, and then, it was fixed to 4% PFA (paraformaldehyde). After paraffin embedding, it was sectioned into 5 μm, and then attached to a slide, and then immunohisto-fluorescence staining was performed, and the antibody was stained using the TRPV1 marker, a pain marker, and the NeuN marker for staining the nucleus of nerve cells. mouse anti-TRPV1 and rabbit anti-NeuN were used as primary antibodies, and Alexa 488 and Alexa 647 (Invitrogen) were used as secondary antibodies. After staining, it was mounted and photographed with a confocal microscope, and the ROI (region of interest) was 160×160 μm². The TRPV1 expression amount was divided by the NeuN expression amount and quantified, and the results are shown in FIG. 10.

FIG. 10A is a qualitative analysis image and FIG. 10B is a quantitative analysis graph of the degree of recovery through the pain marker TRPV1 of the composition for bone regeneration according to an embodiment; Injury: OVX+injury after four weeks, PMMA: OVX+injury after four weeks+PMMA, PM gel: OVX+injury after four weeks+PM gel (hydrogel:PMMA=8:2), PMMS: OVX+injury after four weeks+PM gel (hydrogel:PMMA=8:2)+spheroids

As shown in FIG. 10A and FIG. 10B, it was confirmed that fluorescence staining is observed in the same or similar amounts for NeuN in all groups, but as for TRPV1, a pain marker, the expression level of fluorescence staining is significantly reduced in the PMMS group compared to the injury and PMMA groups.

Therefore, the PMMS composition for bone regeneration according to an aspect has an effect of not only promoting bone regeneration but also alleviating pain, and thus, can be used as a composition for preventing or treating bone diseases.

The description of the disclosure described above is for illustrative purposes, and a person skilled in the art to which the disclosure appertains will understand that it can be easily modified into other specific forms without changing the technical spirit or essential features of the disclosure. Therefore, it should be understood that the embodiments described above are illustrative in all respects and are not restrictive.

1. A composition comprising:
polymethyl methacrylate (PMMA);
a hydrogel; and
a stem cell spheroid.

2. The composition of claim 1,
wherein the hydrogel comprises glycol chitosan and oxidized hyaluronate,
wherein the glycol chitosan and oxidized hyaluronate are mixed in a weight ratio of 1 to 10:1.

3. The composition of claim 1,
wherein the hydrogel solution and polymethyl methacrylate (PMMA) are mixed in a volume ratio of 1 to 5:1.

4. The composition of claim 1,
wherein the stem cells are composed of human-derived pluripotent stem cells (PSC), embryonic stem cells (ESC), mesenchymal stem cells (MSC), adult stem cells (ASC), induced pluripotent stem cells (iPSC), or a combination thereof.

5. The composition of claim 1,
wherein the stem cells are mesenchymal stem cells (MSC).

6. The composition of claim 1, further comprising an osteoinductive material, an osteoconductive material, an osteogenic material, an osteopromotive material, an anti-osteoporotic material, or an osteophilic material.

7. A pharmaceutical composition for bone regeneration, preventing or treating bone diseases comprising the composition of claim 1.

8. The composition of claim 7, further comprising an osteoinductive material, an osteoconductive material, an osteogenic material, an osteopromotive material, an anti-osteoporotic material, or an osteophilic material.

9. The pharmaceutical composition of claim 7, wherein the composition is injectable into a site where bone disease has progressed.

10. The pharmaceutical composition of claim 7, wherein the composition promotes the growth of cortical bone.

11. The pharmaceutical composition of claim 7,
wherein the bone disease is selected from the group consisting of osteoporosis, compression fracture, lumbar herniated intervertebral disc, degenerative arthritis, rheumatoid arthritis, Paget's disease, osteomalacia, osteosclerosis, and bone tumor.

12. A method for preparing a composition of claim 1,
wherein the method comprises a step for mixing glycol chitosan and oxidized hyaluronate in a weight ratio of 1 to 10:1 to obtain a hydrogel; and
a step for mixing polymethyl methacrylate (PMMA) and a stem cell spheroid in the obtained hydrogel.

13. The method of claim 12, wherein the hydrogel solution and polymethyl methacrylate (PMMA) are mixed in a volume ratio of 1 to 5:1.

14. The method of claim 12, wherein the stem cells are composed of human-derived pluripotent stem cells (PSC), embryonic stem cells (ESC), mesenchymal stem cells (MSC), adult stem cells (ASC), induced pluripotent stem cells (iPSC), or a combination thereof.

15. The method of claim 12, wherein the stem cells are mesenchymal stem cells (MSC).

16. The method of claim 12,
further comprising a step for adding an osteoinductive material, an osteoconductive material, an osteogenic material, an osteopromotive material, an anti-osteoporotic material, or an osteophilic material.

17. A method for promoting bone regeneration, preventing or treating bone diseases comprising:
administering an effective amount of the composition of claim 1.

18. The method of claim 17, wherein the bone disease is selected from the group consisting of osteoporosis, compression fracture, lumbar herniated intervertebral disc, degenerative arthritis, rheumatoid arthritis, Paget's disease, osteomalacia, osteosclerosis, and bone tumor.

19. The method of claim 17, wherein the composition of claim 1 is injected into a site where bone regeneration needs to be promoted or bone disease has progressed.

20. The method of claim 17, wherein the composition of claim 1 further comprises an osteoinductive material, an osteoconductive material, an osteogenic material, an osteopromotive material, an anti-osteoporotic material, or an osteophilic material.
